(19) United States
(12) Patent Application Publication
(10) Pub. No.: US 2010/0217347 A1
(43) Pub. Date: Aug. 26, 2010

(54) NEUROSTIMULATION FOR THE TREATMENT OF PULMONARY DISORDERS

(75) Inventors: John M. Swoyer, Andover, MN (US); Richard M. Farrell, Grant, MN (US)

Correspondence Address: Greatbatch Ltd., 10,000 Wehrle Drive, Clarence, NY, 14031, US

(73) Assignee: GREATBATCH, INC., CLARENCE, NY (US)

(21) Appl. No.: 11/957,710

(22) Filed: Dec. 17, 2007

Related U.S. Application Data
(60) Provisional application No. 60/870,389, filed on Dec. 16, 2006.

(51) Int. Cl. A61N 1/36

(52) U.S. Cl. 607/42; 607/59

(57) ABSTRACT

Uses of a neurostimulator to treat various conditions including asthma, chronic obstructive pulmonary disease (COPD) and allergies is described. A neurostimulator may be used to modulate neural impulses into and out of the target organs, in this case the bronchi and other airway structures. Stimulation can be used to cause airway dilation and also to inhibit airway constriction.

### CROSS-REFERENCE TO RELATED APPLICATION

The present application claims priority from U.S. Provisional Application Ser. No. 60/870,389, filed Dec. 16, 2006.

### BACKGROUND OF THE INVENTION

The present invention relates generally to neurostimulation methods and devices. More specifically, the present invention is related to stimulation of nerves to treat pulmonary conditions. Medical conditions such as asthma, chronic obstructive pulmonary disease (COPD), and allergies could benefit from treatments not requiring the use of administered drugs or pharmaceuticals.

### SUMMARY OF THE INVENTION

The present invention provides an implanted neurostimulator system for the treatment of asthma or other pulmonary diseases. The present invention provides a system designed to cause airway dilation and/or inhibit airway constriction via neuromodulation. One neuromodulation system includes a sensor that creates a “closed loop” system for treating pulmonary disease. Another system stimulates both the sympathetic and parasympathetic systems that innervate the pulmonary system.

These and other objects of the present invention will become increasingly more apparent to those skilled in the art by a reading of the following detailed description in conjunction with the appended drawings.

### BRIEF DESCRIPTION OF THE DRAWINGS

FIG. 1 is a simplified block diagram of an implantable embodiment of an electrical generator suitable for practicing the present invention.

FIG. 2 shows a suitable location of the electric signal generator and electrodes implanted in the patient's body.

FIG. 3 is an illustrative idealized electrical output signal waveform of the signal generator useful for clarifying relevant parameters of the output signal.

### DETAILED DESCRIPTION OF THE PREFERRED EMBODIMENTS

The bronchi and lungs are innervated by the sympathetic division of the nervous system via projections from the cervical spine. They are also innervated by the parasympathetic system primarily through the vagus nerve. Generally, the sympathetic system causes airway dilation and the parasympathetic system causes airway constriction.

Implantable neurostimulators are well known devices, and are currently in use for spine stimulation, gastric stimulation, pain masking, deep brain stimulation, and other uses. The same or similar devices and associated leads may be used to implement the present invention, which includes the use of a neurostimulator to treat various conditions including asthma, chronic obstructive pulmonary disease (COPD), and allergies. Other uses include treatment of rhinorrhea and sialorrhea (excessive salivation). The neurostimulator is used to modulate neural impulses into and out of the target organs, in this case the bronchi and other airway structures.

In general, the sympathetic system causes airway dilation and the parasympathetic system causes airway constriction. Stimulation using some embodiments of the present invention can be applied via the sympathetic system to override reflexes that cause airway constriction. The sympathetic system can be further divided into the afferent and efferent systems. Stimulation of the efferent nerves can cause airway dilation. Stimulation of afferent nerves can cause centers in the brain to modify efferent systems of the sympathetic system also causing airway dilation, or suppression of the parasympathetic system leading to suppression of airway constriction. Stimulation of the parasympathetic system can also be effective; either by modulating or blocking their input into the airways, or causing a feedback loop via afferent nerves that effectively terminates airway constriction. Stimulation targets include the cervical spinal column, nerve roots from the cervical and/or thoracic column and their associated ganglia, the vagus nerve, and the specific nerves at or near their innervation site of the lung and bronchi, including the bronchial ganglion neurons. The stimulator can be programmed to provide constant or intermittent stimulation.

Alternatively, the stimulator can be designed such that an activator remotely initiates stimulation when the patient feels an attack coming on. In that case, a pressure sensor is not needed.

The system can also be designed with a sensor that detects a change in pleural pressure consistent with an attack. Upon sensing an abnormal pressure change, the system automatically turns on stimulation, and then turns it off when pressure is normalized.

Alternatively, the system senses changes in electrical activity or blood chemistry, etc.

Another embodiment of the system modulates the spinal cord in the cervical spine or the spinal roots in the area that is responsible for innervating the bronchi or the lungs. In that case, it is more appropriate to use a lower frequency stimulation in the range of about 2 to about 20 Hz to modulate and restore balance to the control system.

According to one embodiment of the present invention, the vagus nerve is stimulated by delivering an electrical signal generated by any suitable vagus nerve stimulators. The vagus nerve can be stimulated by means of either an implanted device or a device worn external to the patient's body, such as a Cyberonics NCP™ device described in U.S. Pat. No. 5,231,988 or a Medtronic™ device described in U.S. Pat. No. 5,330,507. Both patents describe apparatus for stimulating the right or left vagus nerve with continuous and/or phased electrical signals. Other examples of vagus nerve stimulators are described in U.S. Pat. Nos. 4,702,254; 5,154,172; 5,231,988; 5,330,507; 6,473,644; 6,721,603; 6,735,471; and U.S. Pat. App. Pub. No. 2004/0193231. The teachings of all of these publications are incorporated herein by reference.

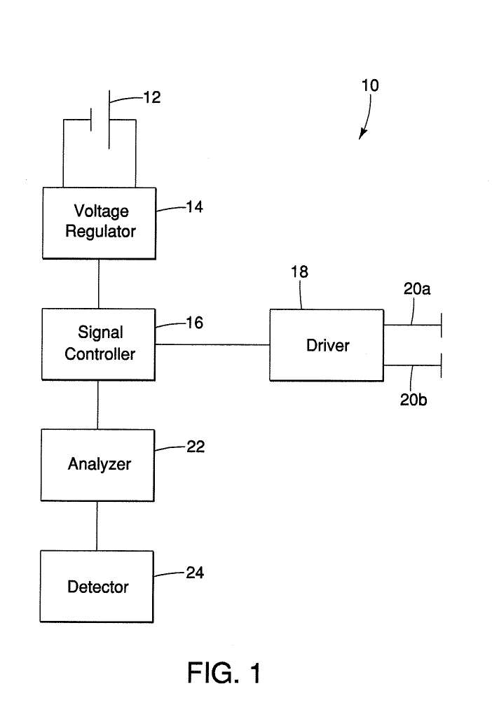

FIGS. 1 to 3 are reproduced from U.S. Patent Publication No. 2006/0178703 to Jared et al., which is incorporation by reference. A schematic diagram of a typical electrical pulse generator device for practicing the present invention is shown in FIG. 1. The pulse generator 10 includes a power source 12, which may be of any type conventionally employed for powering medical electronic devices including primary batteries, rechargeable batteries, and rechargeable capacitors. If the power source is rechargeable, that can be done externally via radio frequency. The power source 12 is connected to a voltage regulator 14. Regulator 14 smoothes the power source output to produce a steady output voltage as well as provide voltage multiplication or division if necessary.

The regulator 14 supplies power to signal controller 16, which can include a microprocessor. The signal controller 16 controls functions of the device such as output signal current or voltage, output signal frequency, output signal pulse width, output signal on-time, and output signal off-time. The controller 16 can be programmed to control daily times for continuous or periodic modulation of vagul activity as well as output signal start delay time. Such programmability allows the output signal to be adjusted for the treatment regimen. Signal controller 16 controls driver 18 which generates the desired electrical signal.

The output signal is applied to the patient's body via electrodes 20a and 20b. The analyzer 22 processes relevant physiological parameters of a patient such as pleural pressure, blood gases, or blood chemistry, that are detected by the detector 24. In one exemplary embodiment, the detector is a pressure sensor that is implanted within the body to sense respiration and changes in pressure associated with an “attack”. The sensor may be attached to a stimulation lead, a separate lead or the pulse generator itself. The sensor may also be a separate unit that communicates wirelessly to the pulse generator.

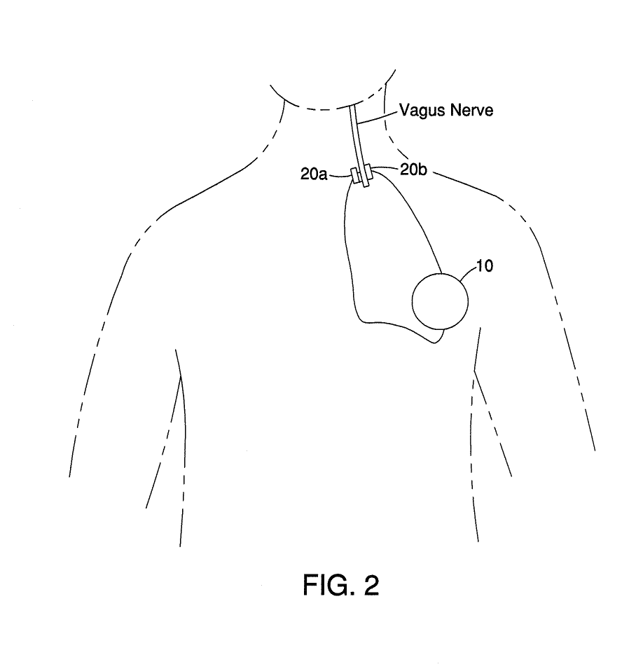

The pulse generator 10 can be worn external to the patient's body or it can be implanted. When the generator 10 is implanted, a built-in antenna (not shown) can be used to enable communication between it and an external programming or monitoring devices (not shown). FIG. 2 illustrates one embodiment of the present invention where pulse generator 10 is implanted in the patient's chest in a pocket formed by the surgeon just below the skin as a pacemaker would be implanted, with the electrodes 20a and 20b implanted in the patient's neck.

The electrodes 20a and 20b can be bipolar stimulating electrodes of the type described in U.S. Pat. No. 4,573,481, incorporated herein by reference. In this embodiment, the electrodes form an assembly which is surgically implanted on the vagus nerve in the patient's neck. The two electrodes may be wrapped around the vagus nerve, and the assembly secured to the nerve by a spiral anchoring tether as disclosed in U.S. Pat. No. 4,979,511, incorporated herein by reference.

In an alternate embodiment, instead of implanting the electrode assembly in the patient's neck, the assembly may be implanted on the vagus nerve as it innervates any of the organs listed above. The implantation of electrodes 20a and 20b may be accomplished in substantially the same manner as described for the neck location. In particular, the pulse generator is implanted in the patient's body, preferably in the upper chest. Stimulating lead electrodes are placed via a cuff electrode on or near the specific projections of the vagus nerve that innervate the bronchi and/or lungs. The lead is preferably multi-polar with at least three electrodes. Typically, for a cuff electrode the center electrode is the cathode and the outer two are anodes.

A sensor lead having a pressure sensor mounted on the distal end is implanted with that the sensor being in the pleural space for monitoring pressure changes associated with respiration. A microprocessor is programmed to distinguish between normal respiration and an asthmatic type of attack. Generally, there will be a greater negative pressure as the patient attempts to inhale during bronchial constriction. The system then stimulates the vagus nerve to terminate the asthmatic attach by blocking parasympathetic input. One type of signal that has been shown to block conduction is of a high frequency signal greater than about 50 Hz, preferably about 130 Hz. Typical amplitudes are in the range of about 0.5 volts to about 12 volts, preferably about 2 to about 7 volts. The signal is applied until the system senses that the attack has terminated.

The system could also have a patient activated controller that is used to manually initiate stimulation, manually terminate stimulation, turn off or otherwise adjust the implanted system via telemetry. The patient controller could also be used in conjunction with a “smart system” that is taught to identify an asthma attack. In that case, the patient would signal the implantable pulse generator system when an attack starts and ends, allowing the system to “learn” the appropriate physiological signals specific to the patient. This requires programming the controller to activate and de-activate based on a particular patient's unique physiological conditions as the patient manually activates and de-activates the controller upon the onset and end of an asthma attack. As the number of attacks increases, the controller compiles that data into a database specific to a particular patient's physiological condition to, in effect, anticipate the onset and end of an asthma episode.

The lead for vagus nerve stimulation may be a cuff style lead with one or more electrodes, preferably three. Alternatively, the lead may consist of one or more electrodes placed near the vagus nerve. The electrodes may be cylindrical, preferably directional, such that current is directed towards the stimulation site and not towards unintended targets. Examples include partially insulated cylindrical electrodes or plate electrodes. This lead could be one electrode or more, preferably four to eight electrodes. Ideally, the lead is a self-anchoring type that implants near the target nerve via tines or fins.

Spinal cord stimulation leads are preferably either paddle type leads or cylindrical type leads. Such leads are commonly used for SCS for pain.

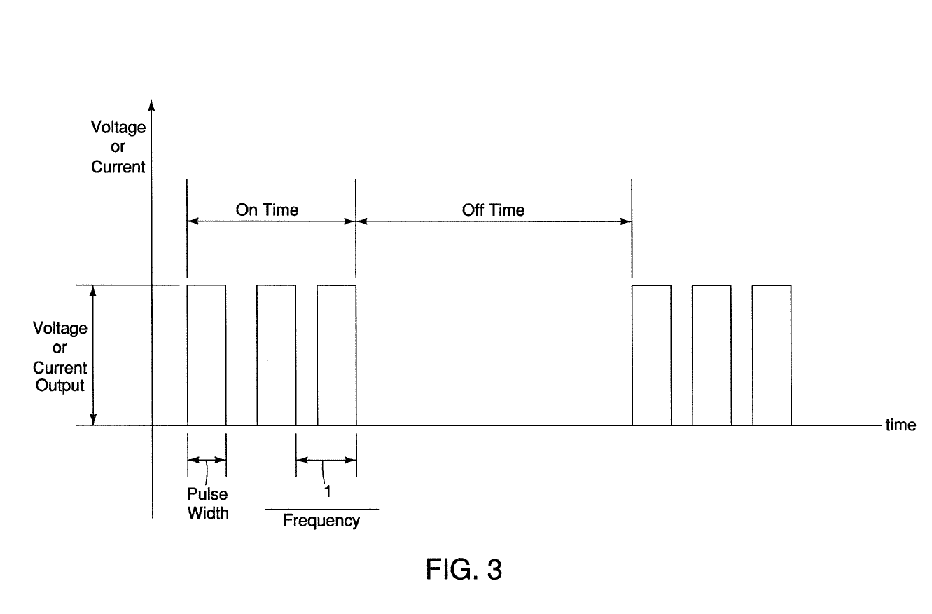

Operation of the pulse generator 10 to control and treat pulmonary disorders may be described by reference to the signal waveform and parameters shown in FIG. 3. The drawing is an idealized representation of the output signal delivered by driver 18 and serves to clarify terminology used to refer to the parameters of an electrical signal. Such parameters include signal on-time, signal off-time, signal frequency, signal pulse width, signal current, and signal voltage. Treatment of pulmonary disorders can be accomplished by applying voltage to electrodes 20a and 20b as well as by driving a current between electrodes 20a and 20b. While the pulses shown in FIG. 3 have positive voltage or current output, electrical signals having negative outputs can also be used.

It is appreciated that various modifications to the inventive concepts described herein may be apparent to those of ordinary skill in the art without departing from the scope of the present invention as defined by the appended claims.

1. A pulse generator adapted for delivering a therapy to a body tissue to treat a condition selected from asthma, chronic obstructive pulmonary disease, rhinorrhea, sialorrhea, and allergies.

2. The pulse generator of claim 1 comprising:
a) an electrical power source;
b) a voltage regulator connected to the electrical power source for providing an output voltage;
c) a controller connected to the voltage regulator to provide an output signal; and
d) a driver for generating a desired electrical output from the controller to at least one lead connected to electrodes implanted in a body tissue intended to be treated.

3. The pulse generator of claim 1 wherein the controller is programmed to distinguish between normal respiration and bronchial constriction.

4. The pulse generator of claim 1 wherein the controller regulates at least one of the group consisting of an output signal current, an output voltage, an output signal frequency, an output signal pulse width, an output signal on-time, and an output signal off-time.

5. The pulse generator of claim 1 wherein electrodes are implanted on the vagus nerve in a patient's neck.

6. The pulse generator of claim 5 wherein the electrical output is at a voltage ranging from about 5 volts to about 12 volts at a frequency greater than about 50 Hz.

7. The pulse generator of claim 1 wherein electrodes are implanted in the cervical spine or the spinal root.

8. The pulse generator of claim 7 wherein the electrical output is at a voltage ranging from about 5 volts to about 12 volts at a frequency in a range of about 2 Hz to about 20 Hz.

9. The pulse generator of claim 1 wherein the controller is manually activatable by the patient.

10. The pulse generator of claim 9 wherein the manually activatable controller is programmable to a specific patient's physiological conditions regarding normal respiration and bronchial constriction.

11. The pulse generator of claim 1 being at least partially external of the body,

12. The pulse generator of claim 1 being implanted in the body.

13. The pulse generator of claim 1 at least partially implanted in a chest of the body.

14. The pulse generator of claim 1 wherein the electrode are either bipolar or multi-polar.

15. The pulse generator of claim 1 wherein the electrodes are implanted on the vagus nerve as it inervates an organ selected from the group consisting of the cervical spinal column, nerve roots from the cervical column and associated ganglia, nerve roots from the thoracic column and associated ganglia, specific nerves at or near the innervation site of the lungs, and specific nerves at or near the innervation site of the bronchi, bronchial ganglion neurons, and combinations thereof.

16. The pulse generator of claim 1 further including an analyzer connected to a detector for processing physiological parameters of the body in response to the delivered therapy.

17. A method for treating a condition selected from asthma, chronic obstructive pulmonary disease, rhinorrhea, sialorrhea, and allergies, the method comprising:
a) providing a closed loop stimulation system, such that the stimulation system is responsive to physiologically originated signals;
b) providing a sensor, that is incorporated into the closed loop stimulation system, such that the sensor detects a change in pleural pressure, that upon detecting an abnormal pressure change, the system automatically turns on stimulation, and then turns the stimulation off when pressure is normalized by emitting the physiologically originated signal; and
c) using the sensor to stimulate a target portion of the nervous system to control dilation of at least one of the lungs and bronchi.

18. The method of claim 17 including stimulating the vagus nerve.

19. The method of claim 17 including providing the stimulation being substantially constant.

20. The method of claim 17 including providing the stimulation being intermittent.

21. The method of claim 17 including providing the stimulation increasing airway dilation.

22. The method of claim 17 including providing the stimulation inhibiting airway constriction.

23. (canceled)

24. The method of claim 17 including stimulating a target portion of the nervous system selected from the group consisting of: the cervical spinal column, nerve roots from the cervical column and associated ganglia, nerve roots from the thoracic column and associated ganglia, specific nerves at or near the innervation site of the lungs, and specific nerves at or near the innervation site of the bronchi, bronchial ganglion neurons, and combinations thereof.

25. The method of claim 17 including stimulating efferent nerves to effect airway dilation.

26. The method of claim 17 including stimulating afferent nerves to effect airway dilation or suppression of airway constriction.

27. The method of claim 17 including distinguishing between normal respiration and bronchial constriction.

28. The method of claim 17 including regulating at least one of the group consisting of an output signal current, an output voltage, an output signal frequency, an output signal pulse width, an output signal on-time, and an output signal off-time.

29. The method of claim 17 including implanting electrodes on the vagus nerve in a patient's neck as part of the closed loop stimulation system.

30. The method of claim 29 including providing an electrical output ranging from about 5 volts to about 12 volts at a frequency greater than about 50 Hz.

31. The method of claim 17 including implanting electrodes in the cervical spine or the spinal root as part of the closed loop stimulation system.

32. The method of claim 31 including providing an electrical output ranging from about 5 volts to about 12 volts at a frequency in a range of about 2 Hz to about 20 Hz.

33. (canceled)

34. (canceled)

35. (canceled)

36. The method of claim 17 including implanting electrodes as part of the closed loop stimulation system on the vagus nerve as it inervates an organ selected from the group consisting of the cervical spinal column, nerve roots from the cervical column and associated ganglia, nerve roots from the thoracic column and associated ganglia, specific nerves at the innervation site of the lungs, and specific nerves at the innervation site of the bronchi, bronchial ganglion neurons, and combinations thereof.
